(19) United States
(12) Patent Application Publication
(10) Pub. No.: US 2016/0331583 A1
(43) Pub. Date: Nov. 17, 2016

(54) RIGID CONTAINER FOR DISTRIBUTING COOLING TEMPERATURES TO LIMBS

(71) Applicant: Sheldon Geringer, Aiea, HI (US)

(72) Inventor: Sheldon Geringer, Aiea, HI (US)

(21) Appl. No.: 14/709,326

(22) Filed: May 11, 2015

(51) Int. Cl. A61F 7/10; A61F 7/02

(52) U.S. Cl. CPC A61F 7/103; A61F 7/02; A61F 2007/003

(57) ABSTRACT

Some embodiments of the present disclosure include a container for cooling a portion of a user's body. The container may include an outer shell with an inner cavity configured to accommodate a volume of fluid, the outer shell comprising at least one limb slot configured to accommodate a user's limb and a fill spout extending from the outer shell, wherein the fill spout is configured to provide an opening into the inner cavity into which the fluid can be poured. The fluid may be any suitable fluid that can be cooled using conventional cooling devices.

### BACKGROUND

The embodiments herein relate generally to medical devices, and more particularly, to a rigid container configured to accommodate cold or frozen fluids for distributing cooling temperatures to a user's limbs.

Common cooling methods after exercise or injury, such as the use of ice or freezable compresses are sloppy and potentially burn the skin. Specifically, although ice wrapped in a towel provides cooling, the ice melts and causes a mess. Moreover, ice is not always available and takes time to freeze. Furthermore, ice can irritate or burn the skin and does not provide even cooling, since it is composed of irregular shaped cubes. Soft gel packs are thin and tend to have the same burning/irritation affect as ice on exposed skin.

Therefore, what is needed is a container configured to accommodate a volume of cold or frozen fluids, wherein the container comprises a surface that accommodates a user's limb.

### SUMMARY

Some embodiments of the present disclosure include a container for cooling a portion of a user's body. The container may include an outer shell with an inner cavity configured to accommodate a volume of fluid, the outer shell comprising at least one limb slot configured to accommodate a user's limb and a fill spout extending from the outer shell, wherein the fill spout is configured to provide an opening into the inner cavity into which the fluid can be poured. The fluid may be any suitable fluid that can be cooled using conventional cooling devices.

### BRIEF DESCRIPTION OF THE FIGURES

The detailed description of some embodiments of the invention is made below with reference to the accompanying figures, wherein like numerals represent corresponding parts of the figures.

FIG. 1 is a perspective view of one embodiment of the present disclosure, shown in use.

FIG. 2 is a perspective view of one embodiment of the present invention.

FIG. 3 is a front view of one embodiment of the present invention.

FIG. 4 is an exploded view of one embodiment of the present disclosure.

FIG. 5 is a section view of one embodiment of the present invention, taken along line 5-5 in FIG. 1.

FIG. 6 is a section view of one embodiment of the present invention, taken along line 6-6 in FIG. 5.

FIG. 7 is a perspective view of one embodiment of the present invention.

FIG. 8 is a section view of one embodiment of the present invention, taken along line 8-8 in FIG. 7.

FIG. 9 is a section view of one embodiment of the present invention, taken along line 9-9 in FIG. 8.

### DETAILED DESCRIPTION OF CERTAIN EMBODIMENTS

In the following detailed description of the invention, numerous details, examples, and embodiments of the invention are described. However, it will be clear and apparent to one skilled in the art that the invention is not limited to the embodiments set forth and that the invention can be adapted for any of several applications.

The device of the present disclosure may be used to distribute cooling temperatures to a user's limb and may comprise the following elements. This list of possible constituent elements is intended to be exemplary only, and it is not intended that this list be used to limit the device of the present application to just these elements. Persons having ordinary skill in the art relevant to the present disclosure may understand there to be equivalent elements that may be substituted within the present disclosure without changing the essential function or operation of the device.

1. Outer Shell

2. Inner Cavity

3. Fill Spout

4. Limb Slot

The various elements of the container for distributing cooling temperatures over the limb of a user of the present disclosure may be related in the following exemplary fashion. It is not intended to limit the scope or nature of the relationships between the various elements and the following examples are presented as illustrative examples only.

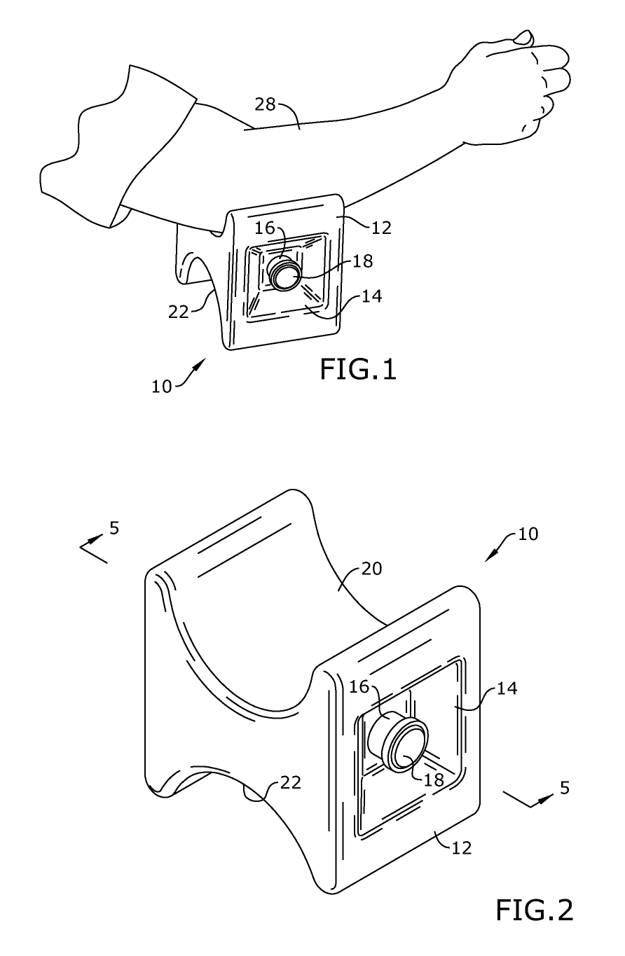

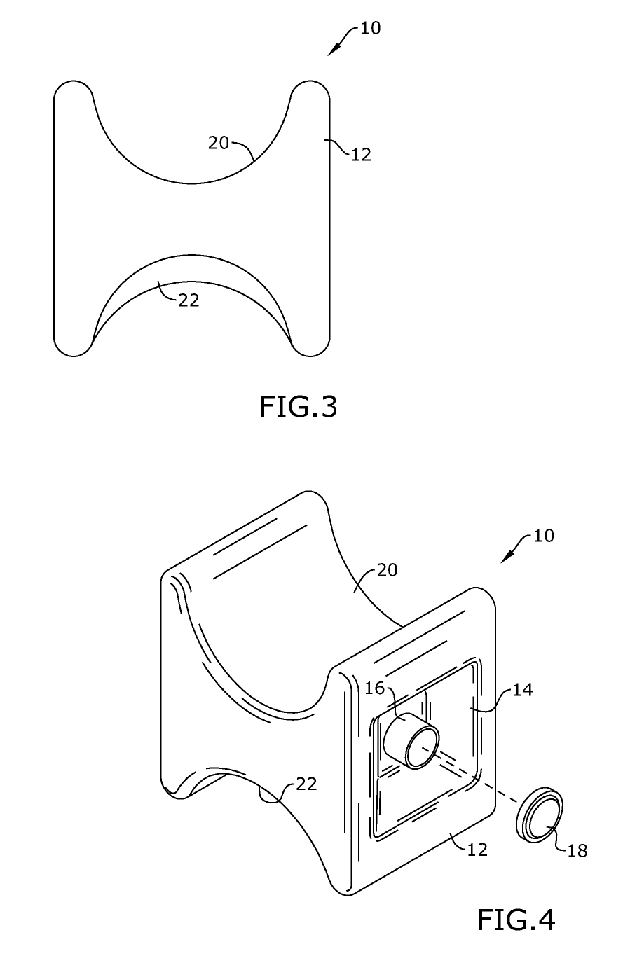

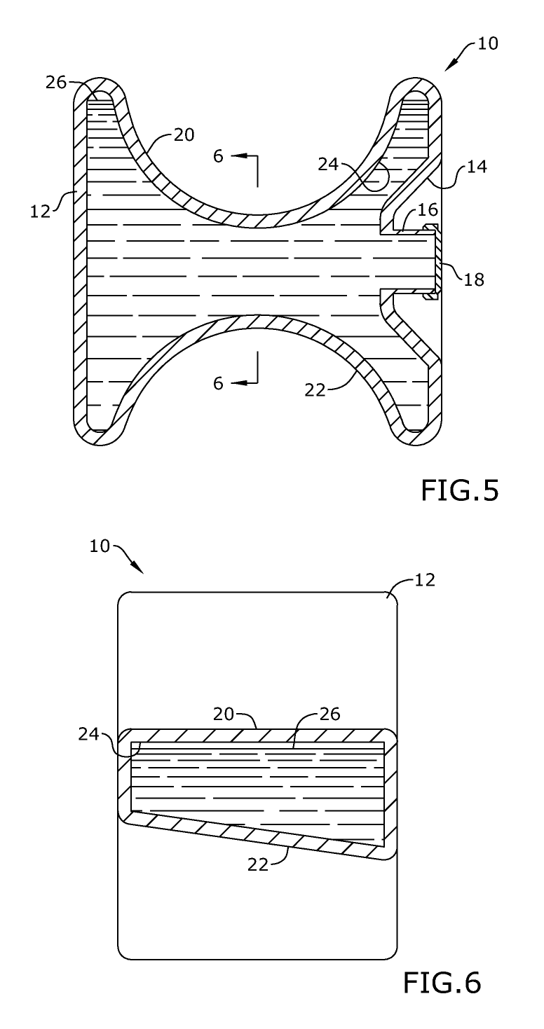

By way of example, and referring to FIGS. 1-9, some embodiments of the container 10 of the present disclosure comprise an outer shell 12 with an inner cavity 24 configured to accommodate a volume of fluid 26, wherein the outer shell 12 comprises at least one limb slot configured to accommodate a user's limb 28, and a fill spout 16 extending from the outer shell 12, wherein the fill spout 16 provides an opening into the inner cavity 24 into which the fluid 26 can be poured. In embodiments, a spout cavity 24 may be positioned around the fill spout 16. The spout cavity 24 may function to collect fluid 26 that is being poured into the fill spout 16 but that misses the fill spout 16. The container may further comprise a cap 18 configured to seal the fill spout 16 closed such that fluid 26 does not flow out of the inner cavity 24.

In some embodiments, and as shown in FIGS. 1-6, the container 10 of the present disclosure may comprise a plurality of limb slots, such as a first limb slot 20 and a second limb slot 22. As shown in the Figures, the first limb slot 20 may have a different shape than the second limb slot 22. For example, the first limb slot 20 may have a straight cross section, as shown in FIG. 6, while the second limb slot 22 may have a sloped cross section. The varying cross sections may accommodate different limbs more comfortably. For example, the first limb slot 20 may be configured to accommodate a user's arm 28, while the second limb slot 20 may have a shape more suitable for accommodating a user's leg or knee. When the container 10 includes both the first limb slot 20 and the second limb slot 22, the container 10 may be configured to simultaneously accommodate a user's arm 28 and knee.

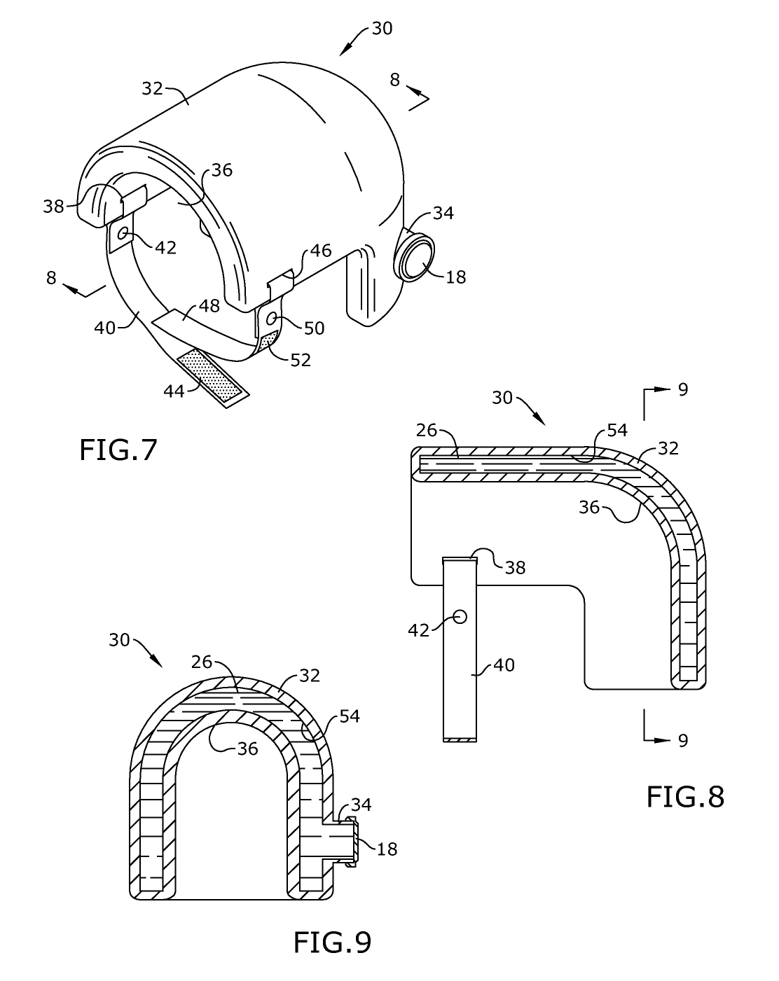

As shown in FIGS. 7-8, embodiments of alternate containers 30 may be configured another portion of a user's body, such as a user's shoulder. In such embodiments, the container 30 may comprise an outer shell 32 comprising a shoulder slot 36 that is shaped to accommodate a user's shoulder and rotator cuff, wherein the outer shell 32 defines an inner cavity 54 configured to accommodate a volume of fluid 26. As with the earlier described container 10, the alternate container 30 may have a fill spout 34 with a cap extending from the outer shell 54 such that fluid 26 may be placed into the inner cavity 54. A securing device may be positioned proximal to the outer edge of the shoulder slot 36.

In some embodiments, the securing device may comprise a first strap 40 configured to removably engage with a second strap 48, wherein the straps 40, 48 are configured to wrap around the user's armpit, securing the container 30 in place. The first strap 40 may extend from a first strap slot 38 in the outer shell 32, and the second strap 48 may extend from a second strap slot 46 in the outer shell 32. A first strap post 42 may secure the end of the first strap 40 proximal to the first strap slot 38, such that the first strap 40 is securely attached to the outer shell 32. Similarly, a second strap post 50 may secure the end of the second strap 48 proximal to the second strap slot 46, such that the second strap 48 is securely attached to the outer shell 32. The straps 40, 48 may include complimentary fasteners configured to engage with one another such that the straps 40, 48 may be wrapped around a user's body, such as the armpit, and secured. In some examples, and as shown in FIG. 7, the fastener may comprise hook fastener 44 attached to the first strap 40 and loop fastener 52 attached to the second strap 52, wherein the hook and loop fasteners 44, 52 may adjustably engage to secure the container 30 to a user's limb.

In any embodiment, a volume of fluid may be placed into the inner cavity of the container, and the container may be cooled by placing it in, for example, a refrigerator or freezer. Once the fluid in the container is sufficiently cooled, the container may be positioned to accommodate a user's body part, such as an arm, leg, shoulder, or the like. When needed, the container may be secured to the user's body using the securement straps.

The container of the present disclosure may be made of any suitable material and, in some embodiments, comprises a rigid material configured to retain its shape. For example, the container may comprise a plastic material. Similarly, the fluid may comprise any desired fluid, such as a liquid, like water, or a gel.

Persons of ordinary skill in the art may appreciate that numerous design configurations may be possible to enjoy the functional benefits of the inventive systems. Thus, given the wide variety of configurations and arrangements of embodiments of the present invention the scope of the invention is reflected by the breadth of the claims below rather than narrowed by the embodiments described above.

What is claimed is:

1. A container for cooling a portion of a user's body, the container comprising:
an outer shell with an inner cavity configured to accommodate a volume of fluid, the outer shell comprising at least one limb slot configured to accommodate a user's limb; and
a fill spout extending from the outer shell, wherein the fill spout is configured to provide an opening into the inner cavity into which the fluid can be poured.

2. The container of claim 1, further comprising a spout cavity positioned around the fill spout.

3. The container of claim 1, further comprising a cap configured to seal the fill spout closed such that the fluid does not flow out of the inner cavity when the container is in use.

4. The container of claim 1, wherein the container comprises:
a first limb slot configured to accommodate a first limb; and
a second limb slot configured to accommodate a second limb, such that the container is configured to simultaneously accommodate the first limb and the second limb.

5. The container of claim 4, wherein first limb slot has a different shape than the second limb slot.

6. The container of claim 5, wherein:
the first limb slot has a straight cross section; and
the second limb slot has a sloped cross section.

7. The container of claim 1, wherein the outer shell comprises:
a shoulder slot that is shaped to accommodate a user's shoulder and rotator cuff; and
a securing device positioned proximal to an outer edge of the shoulder slot, the securing device configured to secure the container to the user's shoulder and rotator cuff.

8. The container of claim 7, wherein the securing device comprises a first strap configured to removably engage with a second strap, the straps being configured to wrap around the user's armpit, securing the container in place.

9. The container of claim 8, wherein:
the first strap extends from a first strap slot in the outer shell;
the second strap extends from a second strap slot in the outer shell;
a first strap post secures the first strap proximal to the first strap slot; and
a second strap post secures the second strap proximal to the second strap slot.

10. The container of claim 1, wherein the container comprises a rigid material configured to retain its shape while in use.
